(19) United States
(12) Patent Application Publication
(10) Pub. No.: US 2022/0379724 A1
(43) Pub. Date: Dec. 1, 2022

(54) DEVICES, METHODS AND SYSTEMS RELATED TO WEARABLE PATCH HAVING BLOOD ALCOHOL CONTENT DETECTOR

(71) Applicant: LIFE PATCH INTERNATIONAL, Irvine, CA (US)

(72) Inventors: Brigitte Chau PHAN, Irvine, CA (US); Donald BOLLELLA, Irvine, CA (US)

(21) Appl. No.: 17/885,272

(22) Filed: Aug. 10, 2022

Related U.S. Application Data
(63) Continuation of application No. PCT/US2021/017723, filed on Feb. 11, 2021.
(60) Provisional application No. 62/972,655, filed on Feb. 11, 2020.
(60) Provisional application No. 62/972,656, filed on Feb. 11, 2020.

(51) Int. Cl. B60K 28/06; A61B 5/00; A61B 5/145; H04B 5/00

(52) U.S. Cl. CPC B60K 28/063; A61B 5/6833; A61B 5/74; A61B 5/4845; A61B 5/0002; A61B 5/145; H04B 5/0062; A61B 2562/08; G08B 7/06

(57) ABSTRACT

Devices, methods and systems related to wearable patch having blood alcohol content detector. In some embodiments, a wearable patch can include a patch structure having one or more layers and configured to allow the patch to be worn by a user, and an analyzer component implemented on or at least partially within the patch structure and configured to measure alcohol content level in the user. The wearable patch can further include an interface component implemented on or at least partially within the patch structure and in communication with the analyzer component, with the interface component being configured to provide a notification based on the measured alcohol content. In some embodiments, such a wearable patch can be a part of a system such as a monitoring system or a compliance system.

### CROSS-REFERENCE TO RELATED APPLICATION(S)

This application is a continuation of International Application No. PCT/US2021/017723 filed Feb. 11, 2021, entitled DEVICES, METHODS AND SYSTEMS RELATED TO WEARABLE PATCH HAVING BLOOD ALCOHOL CONTENT DETECTOR, which claims priority to U.S. Provisional Application Nos. 62/972,655 filed Feb. 11, 2020, entitled DEVICES, METHODS AND SYSTEMS RELATED TO BIO-PATCH HAVING BLOOD ALCOHOL CONTENT DETECTOR, and 62/972,656 filed Feb. 11, 2020, entitled COMPLIANCE SYSTEMS HAVING WEARABLE PATCH, the benefits of the filing dates of which are hereby claimed and the disclosures of which are hereby expressly incorporated by reference herein in their entirety.

### BACKGROUND

### Field

The present disclosure relates to wearable patches having blood alcohol content detector and related devices, methods and systems.

### Description of the Related Art

When a person consumes alcohol, he/she can become impaired for certain activities such as operation of vehicles. In many situations, blood alcohol content level provides a measure of such a person's impaired state.

### SUMMARY

In accordance with some implementations, the present disclosure relates to a wearable patch that includes a patch structure having one or more layers and configured to allow the patch to be worn by a user, and an analyzer component implemented on or at least partially within the patch structure and configured to measure alcohol content level in the user. The wearable patch further includes an interface component implemented on or at least partially within the patch structure and in communication with the analyzer component. The interface component is configured to provide a notification based on the measured alcohol content.

In some embodiments, the patch structure can be configured to allow the patch to be worn on or near a skin of the user. The patch structure can be configured to allow the patch to be worn directly on the skin of the user.

In some embodiments, the interface component can be configured to alert either or both of the user and another person when the measured alcohol content exceeds a selected level. In some embodiments, the interface component can include a communication circuit configured to send information about the measured alcohol content. In some embodiments, the communication circuit can be configured to receive information. In some embodiments, the communication circuit can be configured to send the information in a wireless manner. In some embodiments, the communication circuit can be configured to send the information to an external device.

In some embodiments, the alcohol content of the user can include a blood alcohol content (BAC) of the user.

In some implementations, the present disclosure relates to a kit for monitoring alcohol content of a user. The kit includes a wearable patch that includes a patch structure having one or more layers and configured to allow the patch to be worn by the user. The wearable patch further includes an analyzer component implemented on or at least partially within the patch structure and configured to measure alcohol content level in the user. The wearable patch further includes an interface component implemented on or at least partially within the patch structure and in communication with the analyzer component. The interface component is configured to provide a notification based on the measured alcohol content. The kit further includes a printed instruction configured to facilitate use of the wearable patch.

In some teachings, the present disclosure relates to a system for monitoring alcohol content of a person. The system includes a patch configured to be attached to a skin of the person and obtain a measurement of alcohol content level in the person. The patch is further configured to transmit information representative of the measurement. The system further includes a monitor external to the patch. The monitor is configured to receive the information from the patch through a communication link and generate an output based on the information.

In some embodiments, the communication link between the patch and the monitor can be a direct link implemented with a wireless signal. In some embodiments, the communication link between the patch and the monitor can include an intermediate component such that a first link provides communication between the patch and the intermediate component and a second link provides communication between the intermediate component and the monitor. For example, the first link between the patch and the intermediate component can be implemented with a wireless signal, and the second link between the intermediate component and the monitor can be implemented with a wireless signal.

In some embodiments, the patch can include a radio-frequency identification (RFID) circuit configured to receive an interrogation signal and transmit the information in response to receipt of the interrogation signal.

In some embodiments, the system can further include a vehicle electronic control unit configured to receive the output of the monitor and either enable or disable an operation of a corresponding vehicle. The operation of the vehicle can include starting of an internal combustion engine or energizing of an electric drive motor. The output of the monitor can be based on the measured alcohol content level. The output of the monitor can be based on an estimation based on a plurality of measured alcohol content level values.

In some embodiments, the output of the monitor can be based on determination of whether or not the patch remains attached to the skin of the person. In some embodiments, the patch can be configured to sense detachment of the patch from the skin of the person. In some embodiments, the determination of whether or not the patch remains attached to the skin of the person can be based on a comparison of a measured value of alcohol content level with an expected value.

According to some implementations, the present disclosure relates to a system for monitoring impairment state of a person. The system includes a patch configured to be attached to a skin of the person and obtain a measurement indicative of an impairment state of the person. The patch is further configured to transmit information representative of the measurement. The system further includes a monitor external to the patch. The monitor is configured to receive the information from the patch through a communication link and generate an output based on the information.

In some embodiments, the communication link between the patch and the monitor can be a direct link implemented with a wireless signal. In some embodiments, the communication link between the patch and the monitor can include an intermediate component such that a first link provides communication between the patch and the intermediate component and a second link provides communication between the intermediate component and the monitor. For example, the first link between the patch and the intermediate component can be implemented with a wireless signal, and the second link between the intermediate component and the monitor can be implemented with a wireless signal.

In some embodiments, the patch can include a radio-frequency identification (RFID) circuit configured to receive an interrogation signal and transmit the information in response to receipt of the interrogation signal.

In some embodiments, the system can further include a vehicle electronic control unit configured to receive the output of the monitor and either enable or disable an operation of a corresponding vehicle. The operation of the vehicle can include starting of an internal combustion engine or energizing of an electric drive motor.

In some embodiments, the output of the monitor can be based on the measurement. In some embodiments, the output of the monitor can be based on an estimation based on a plurality of measured values related to the impairment state of the person. In some embodiments, the output of the monitor can be based on determination of whether or not the patch remains attached to the skin of the person. In some embodiments, the patch can be configured to sense detachment of the patch from the skin of the person. In some embodiments, the determination of whether or not the patch remains attached to the skin of the person can be based on a comparison of a measured value related to the impairment state of the person with an expected value.

In some embodiments, the measurement can include a blood alcohol content (BAC) level.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 depicts a wearable patch having a blood alcohol content (BAC) analyzer.

FIG. 2 depicts a side view of a wearable patch having one or more features as described herein.

FIG. 3 shows an example of a process that can be implemented with a patch having one or more features as described herein.

FIG. 4 shows another example of a process that can be implemented with a patch having one or more features as described herein.

FIGS. 5A and 5B show a patch that can be an example of the patch described herein in reference to FIGS. 1 to 4.

FIG. 6 shows that in some embodiments, a patch having one or more features as described herein can be implemented in a bracelet format.

FIG. 7 shows that in some embodiments, a patch having one or more features as described herein can be implemented in a patch format.

FIG. 8 shows an example of a patch that can be configured to sample a flow of blood.

FIG. 9 shows an example of a system that can be implemented to utilize a communication functionality.

FIG. 10 shows an example of a system that can be implemented to utilize transmit and receive functionalities.

FIG. 11 shows that in some embodiments, a communication component of a patch can be configured to provide a wireless communication with an external device.

FIG. 12 shows that in some embodiments, a communication component of a patch can be configured to provide a wired communication with an external device.

FIG. 13 shows a system that can be formed with one or more patches as described herein, and an external device.

FIG. 14A shows an example of the system of FIG. 13 utilizing a wearable device and an external device such as a smartphone.

FIG. 14B shows another example of the system of FIG. 13 utilizing a wearable device and an external device such as a smartphone.

FIG. 15 shows yet another example of the system of FIG. 13 utilizing a wearable device and an external device such as a smartphone.

FIGS. 16A to 16C show examples where the system of FIG. 15 can provide appropriate notices to a user regarding his/her impairment state detected by a patch as described herein.

FIG. 17 shows that in some embodiments, the systems of FIGS. 14 to 16 can further include a feature where the smartphone communicates with another smartphone to provide a notice or a request.

FIG. 18 shows that in some embodiments, the system of FIG. 10 can include a plurality of patches that communicate with a common external device.

FIG. 19 shows that in some embodiments, the system of FIG. 10 can include a plurality of patches that can communicate with each other, and/or with an external device.

FIG. 20 shows an example system where a driver is wearing a patch having one or more features as described herein.

FIG. 21 shows an example of a compliance logic circuit that can be implemented in the vehicle electronic control unit of FIG. 13.

FIGS. 22A to 22D show non-limiting examples of one or more intermediate compliance devices that can be implemented in the system of FIG. 13.

FIG. 23 shows that in some embodiments, one or more patches having one or more features as described herein can be provided in a packaged format for easier use.

FIG. 24 shows an example of a packaged format having a support sheet with a plurality of patches secured thereto.

FIG. 25 shows an enlarged side sectional view of an example support sheet that can be utilized to hold a plurality of patches, similar to the example of FIG. 17.

FIG. 26 depicts a patch having one or more features as described herein being applied to a user.

FIGS. 27A and 27B show an example where authentication of a patch user, or driver authentication, can be determined.

FIG. 28 depicts an operation of a system that utilizes a patch having one or more features as described herein.

FIG. 29 shows that in some embodiments, a patch and/or a related system can be configured to predict or estimate a BAC level at a time away from a measurement time.

FIGS. 30A and 30B show examples of how driver authentication can be achieved.

FIG. 31 shows an example of a communication link between a patch and a smartphone for the system of FIG. 22D.

FIG. 32 shows an example of a communication link between a smartphone and one or more key fobs for the system of FIG. 22D.

FIG. 33 shows an example of a signal being sent a smartphone to a key fob to disable operation of the corresponding vehicle.

FIG. 34 shows that in some embodiments, the system of FIG. 22D can include test and/or diagnostic functionality.

FIG. 35 shows an example of an alcohol consumption situation where the system of FIG. 22 can be beneficial and useful.

FIG. 36 shows an example of how a vehicle can be disabled upon detection of impairment due to the alcohol consumption situation of FIG. 35.

### DETAILED DESCRIPTION OF SOME EMBODIMENTS

The headings provided herein, if any, are for convenience only and do not necessarily affect the scope or meaning of the claimed invention.

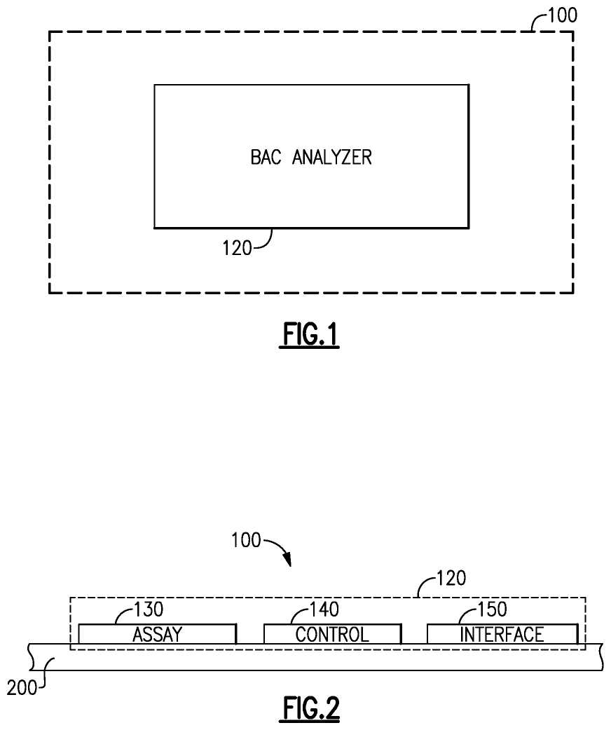

FIG. 1 depicts a wearable patch 100 having a blood alcohol content (BAC) analyzer 120. Such a BAC analyzer can be configured to provide one or more functionalities as described herein. For the purpose of description, the wearable patch 100 may also be referred to herein as a bio-patch, a BAC patch, a drunk driving prevention patch, etc., or simply as a patch.

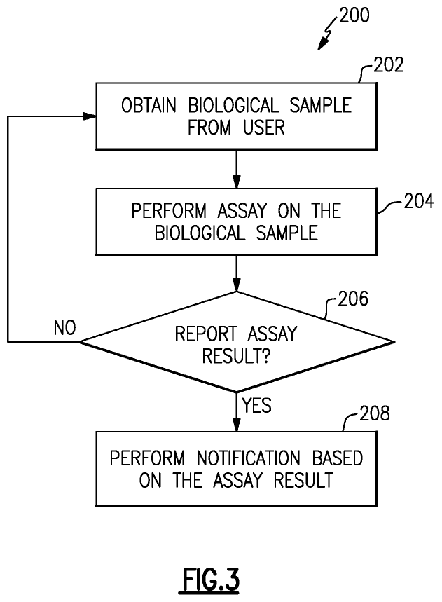

FIG. 2 depicts a side view of a wearable patch 100 having one or more features as described herein. In some embodiments, the wearable patch 100 can include a patch layer 200 configured to allow implementation of one or more functionalities as described herein, and to allow the wearable patch 100 to be worn by a user. Among others, examples related to such a patch layer can be found in U.S. Pat. No. 9,133,024 titled PERSONAL DIAGNOSTIC DEVICES INCLUDING RELATED METHODS AND SYSTEMS, which is expressly incorporated by reference in its entirely, and its disclosure is to be considered part of the specification of the present application.

In some embodiments, a BAC analyzer 120 as described herein can include a plurality of components or functional blocks to support various functionalities as described herein. For example, the BAC analyzer 120 can include an assay component 130 configured to perform one or more BAC-related assays. The BAC analyzer 120 can further includes a control component 140 in communication with the assay component 130 to facilitate the one or more assays. The BAC analyzer 120 can further include an interface component 150 in communication with the control component 140. Such an interface component can be configured to, for example, alert the user (wearing the patch 100) of a selected condition detected by the BAC analyzer 120, communicate with an external monitoring device (e.g., with information related to the selected condition detected by the BAC analyzer 120), etc.

Alcohol-impaired driving is a major safety issue. For example, in 2015, over 10,000 people died in alcohol-impaired driving crashes, accounting for nearly ⅓ of all traffic-related deaths in the Unites States.

In some embodiments, a wearable patch having one or more features as described herein can be configured as a personal bio-patch that monitors in real time, or approximately real time, an alcohol level that the user consumes. Such a personal bio-patch can be configured to, for example, provide an alert to an external device such as a mobile phone when the user's blood alcohol content (BAC) reaches a selected level (e.g., 50%) of an allowable or legal limit (e.g., 0.08%). Alternatively, or in addition, such a personal bio-patch can be configured to generate visual, audible and/or tactile alert(s) for the user, the external device, or some combination thereof.

In some embodiments, a personal bio-patch having one or more features as described herein can be configured to provide a more urgent alert to the external device when the user's measured BAC reaches an allowable limit. Alternatively, or in addition, such an urgent alert can include visual, audible and/or tactile alert(s).

In some embodiments, a personal bio-patch having one or more features as described herein can be configured to provide a wireless signal such as a radio-frequency (RF) signal, an optical signal, an acoustic signal, etc. to an external device associated with a vehicle. Such a vehicle can be the user's vehicle or another vehicle capable of recognizing the wireless signal.

By way of an example, an RF signal detectable can be generated by the personal bio-patch, and such a signal can be received and recognized by a circuit in the user's vehicle, similar to a communication arrangement between a key remote device and the vehicle. When the user's measured BAC level reaches or exceeds an allowable limit, the personal bio-patch can send out an alert signal to the vehicle when the individual enters the vehicle. The vehicle can be configured to prevent the user from driving the vehicle. For example, the vehicle's ignition can be disabled. In another example, the vehicle may be started to provide heating or cooling, but may not be put into a moving gear to prevent motion of the vehicle. In yet another example, the alert signal from the personal bio-patch can result in the vehicle key being deactivated such that the vehicle's door does not open (e.g., while the BAC level remains above the limit).

In some embodiments, a personal bio-patch having one or more features as described herein can be configured to provide an alert to a pre-set contact (e.g., a mobile phone number) regarding the user's BAC level. The personal bio-patch can further include a location tracking functionality (e.g., GPS functionality), and/or coordinate with the user's mobile phone having location tracking functionality, to allow the user to be picked up by a sober person associated with the pre-set contact.

In some embodiments, a personal bio-patch having one or more features as described herein can be configured to provide a continuous or approximately continuous monitoring of the user's BAC level. In some embodiments, the personal bio-patch can be configured to monitor the user's BAC level at intervals (e.g., preset time intervals every 4 minutes).

In some embodiments, a personal bio-patch having one or more features as described herein can be configured to provide BAC level monitoring functionality in a non-invasive manner, an invasive manner, or any combination thereof. For example, a non-invasive configuration can include measurement or estimation of BAC level from the user's perspiration. In another example, an invasive configuration can include measurement or estimation of BAC level in blood by measuring changes in a photoethylsmogram (PPG) signal. In yet another example, a personal bio-patch can be configured to monitor BAC level minimally invasively using microneedle sensor arrays.

In some embodiments, a personal bio-patch having one or more features as described herein can be configured to measure BAC level by, or based on, near-infrared (NIR) spectroscopy. In such a configuration, light can be directed into the skin of the user's body part (e.g., forearm), and its reflection can be measured by an NIR spectrophotometer. Since different molecules reflect light differently, the spectrophotometer can determine the difference between, for example, alcohol, water, and other liquid molecules in the skin. Thus, alcohol molecules can be distinguished from the other molecules and can then be quantified.

It is noted that a number of different tests, assays, and/or technologies can be included in a personal bio-patch having one or more features as described herein, to monitor BAC level of the user. For example, in the context of non-invasive detection, alcohol concentration can be measured or estimated based on photoplethysmogram (PPG) signals. A PPG signal can be measured via an LED transmitter that illuminates a body part such as a finger, and a corresponding receiver can measure changes in light property such as intensity. Since a PPG signal contains information about systolic and diastolic blood pressure, such a signal can be used for detection of BAC level.

In another example, BAC level can be measured or estimated by an NIR spectroscopy technique. It is noted that an NIR spectrophotometer can measure or estimate BAC level by identifying molecules based on how they absorb light. Ethanol alcohol oxidizes into carboxylic acid and uses potassium dichromate as an oxidizing agent in a solution of yellow sulfuric acid. With silver nitrate as a catalyst, this reaction typically happens extremely quickly. When the potassium dichromate reacts to the ethanol's oxidization, the chromate ion changes and changes the color intensity of the yellow solution. The spectrophotometer then compares its light absorbance with that of a pure solution. Using this technique, the spectrometer can measure or estimate the amount of alcohol present. Such a measurement or estimate can be output as, for example, a percentage.

As described herein, the foregoing NIR spectroscopy technique can allow alcohol content to be detected in a non-invasive or less invasive manner—for example, through the skin of a user. A light can be directed into the skin of the user (e.g., at a location covered by the personal bio-patch) and its reflection can be measured by the NIR spectrophotometer as described herein. In many applications, the foregoing NIR spectroscopy technique provide accurate results in a short time frame (e.g., under a minute).

FIG. 3 shows an example of a process 200 that can be implemented with a bio-patch having one or more features as described herein. In block 202, a biological sample can be obtained from a user wearing the bio-patch. For the purpose of description, it will be understood that such a biological sample can be obtained in a non-invasive manner, invasive manner, or any combination thereof. In some applications, such a biological sample can include, for example, sweat, blood, etc. In block 204, an assay can be performed on the biological sample obtained from the user. In some embodiments, such an assay can be configured to detect and/or measure or estimate BAC level of the user, including the examples provided herein, to provide one or more functionalities described herein.

Referring to FIG. 3, in a decision block 206, the process 200 can determine whether some or all of the assay results is to be reported. Such a decision can be made based on, for example, the measured BAC level of the user. If the answer in the decision block 206 is No, the process 200 can obtain and assay another sample from the user immediately or after some time. If the answer in the decision block 206 is Yes, the process 200 can perform a notification based on the assay result in block 208. Examples of such a notification are described herein in greater detail.

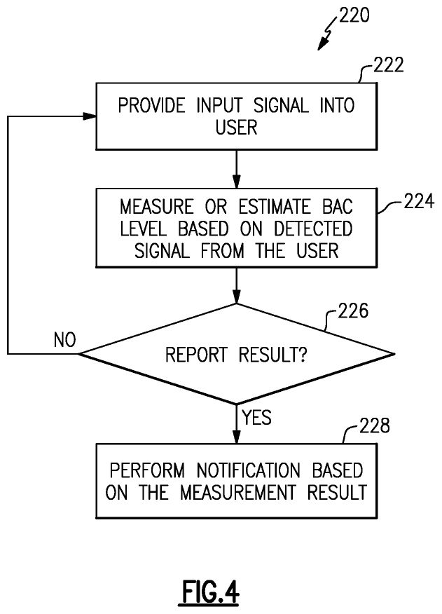

FIG. 4 shows an example of a process 220 that can be implemented with a bio-patch having one or more features as described herein. In block 222, an input signal can be provided to a user by the bio-patch. For the purpose of description, it will be understood that such an input signal can include, for example, an input light utilized for spectroscopy (e.g., NIR spectroscopy). In block 224, a BAC level measurement or estimation can be made for the user based on the input signal.

Referring to FIG. 4, in a decision block 226, the process 220 can determine whether some or all of the measurement results is to be reported. Such a decision can be made based on, for example, the measured BAC level of the user. If the answer in the decision block 226 is No, the process 220 can perform another measurement immediately or after some time. If the answer in the decision block 226 is Yes, the process 220 can perform a notification based on the measurement result in block 228. Examples of such a notification are described herein in greater detail.

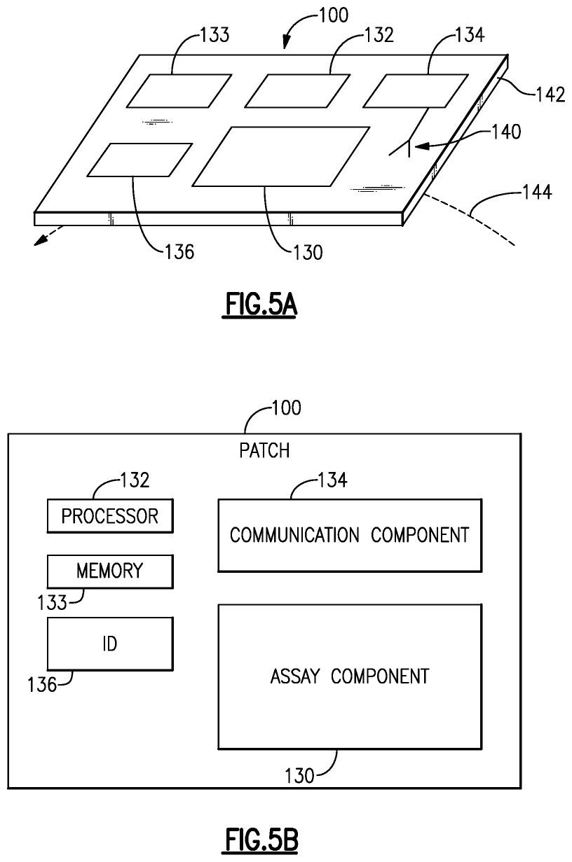

FIGS. 5A and 5B show a patch 100 that can be an example of the bio-patch described herein in reference to FIGS. 1-4. More particularly, the patch 100 of FIGS. 5A and 5B can be configured to include a number of components implemented to allow, for example, one or more BAC-related assays, measurements, etc., and notification of results of such assay(s)/measurement(s) to a monitoring device or system. Accordingly, the patch 100 can include a component 130 configured to perform one or more BAC-related assays/measurements, and a communication component 134 configured to transmit information resulting from the BAC-related assay/measurement. In embodiments where such transmission of information is achieved in a wireless manner, the patch 100 can further include an antenna 140 configured to facilitate the transmission.

In some embodiments, the patch 100 can further include an identifier component 136 configured to provide information about the identity of the patch 100, and therefore, the identity of the user wearing the patch. In some embodiments, information transmitted by the communication component 134 can include the foregoing identifier information.

In some embodiments, some or all of the functionalities associated with the various components of the patch 100 can be controlled and/or facilitated by a processor 132. Similarly, a memory 133 can also be provided to facilitate various functionalities of the patch 100. Such processor and memory can have functionalities as described herein.

In some embodiments, the patch 100 can include a support structure 142 configured to support various components, such as the components shown in FIGS. 5A and 5B. Such a support structure can include one or more layers, and can be implemented in a number of wearable forms. Examples of such a support structure and different wearable forms are provided in the above-referenced U.S. Pat. No. 9,133,024.

In some embodiments, biological fluid such as blood can be obtained by the patch 100, and the BAC-related assay can be performed with such a biological fluid. In some embodiments, such biological fluid can be sampled by the patch 100, where the sampling fluid terminates at the patch 100. In some embodiments, the patch 100 can be configured to allow circulation of biological fluid therethrough, and sampling of the biological fluid for assay purpose can be obtained from such a circulating fluid. In the example of FIG. 5A, such a circulating fluid is depicted as 144.

In some embodiments, non-blood biological fluid such as sweat can be obtained by the patch 100, and the BAC-related assay can be performed with such a biological fluid. In some embodiments, and as described herein, BAC-related measurement can also be achieved by the patch 100, without obtaining of biological fluid from the user. Spectroscopy technique as described herein is an example in which BAC-related measurement can be made without obtaining of biological fluid from the user.

In some embodiments, BAC-related measurement(s) and related devices, methods and/or systems can be implemented in devices that can be worn by a user. Such wearable devices can include patch devices, and many of the examples are described in the context of such patch devices. However, it will be understood that BAC-measurement functionality and devices, methods and/or systems can also be implemented utilizing other wearable devices.

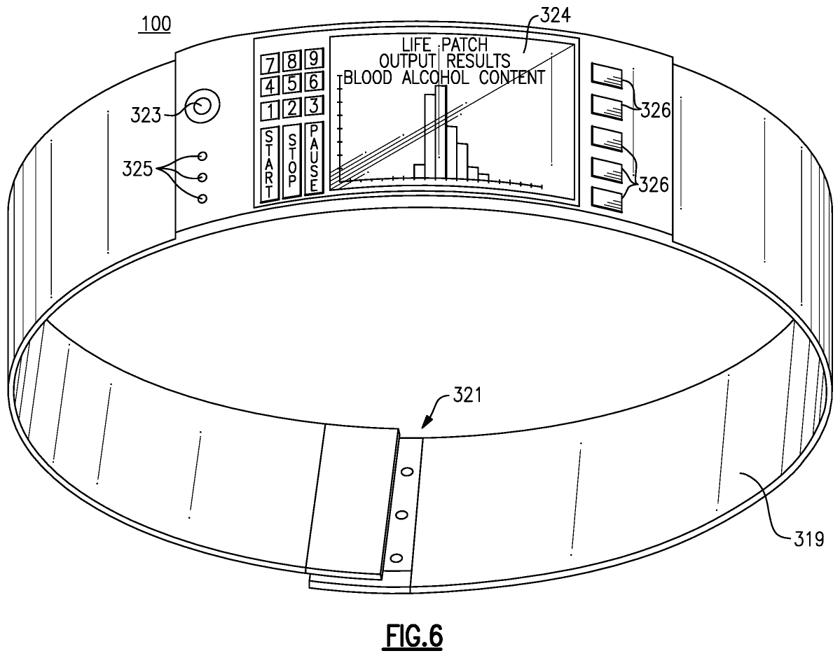

For example, FIG. 6 shows that in some embodiments, a wearable device 100 having one or more features as described herein can be implemented in a bracelet format, and can include a radio frequency transmitter 323 as described herein. The device 100 as shown can also include connection indicator lights 325 which may be enabled to blink or otherwise emit light to indicate a proper wireless connection between the device 100 and an external device.

In the example of FIG. 6, the device 100 can include an RFID circuit configured to provide either or both of receive and transmit functionalities, including some or all of the examples described herein.

The bracelet 100 of FIG. 6 is shown to be provided with a band 319 and a clasp 321 so that a user may wear the band around the wrist or ankle, for example. The band 319 may be made of a flexible, stretch material to provide good holding force between the device and the user, or may be made of a hard material such as stainless steel or aluminum.

FIG. 6 shows that in some embodiments, the device 100 can also include a video display monitor 324 and one or more individual fixed-display results windows 326, implemented to provide information for the user. It is also noted that in the device 100 of FIG. 6, the RF transmitters 323 may include both transmission capabilities as well as receiving capabilities to thereby establish two-way communication between the device 100 and an external device, such as in the examples described herein.

Additional examples related to the device 100 of FIG. 6 can be found in the above-referenced U.S. Pat. No. 9,133,024.

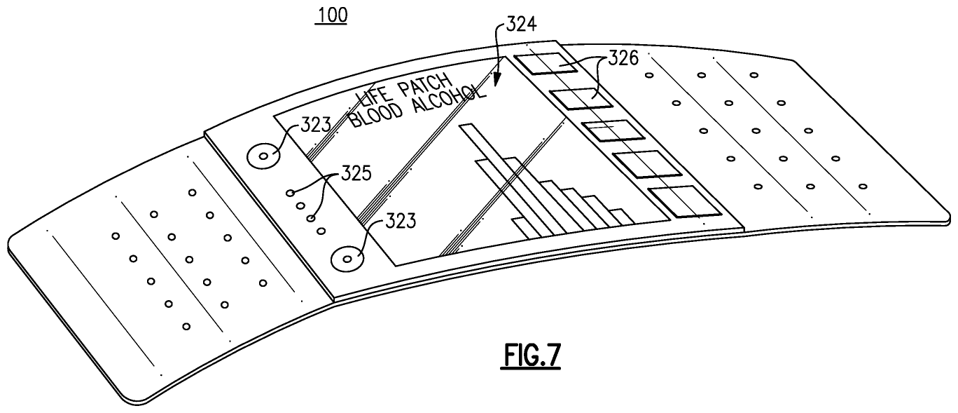

FIG. 7 shows that in some embodiments, a wearable device 100 having one or more features as described herein can be implemented in a patch format, and can include a radio frequency transmitter 323 as described herein. The device 100 as shown can also include connection indicator lights 325 which may be enabled to blink or otherwise emit light to indicate a proper wireless connection between the device 100 and an external device.

In the example of FIG. 7, the device 100 can include an RFID circuit configured to provide either or both of receive and transmit functionalities, including some or all of the examples described herein.

FIG. 7 shows that in some embodiments, the device 100 can also include a video display monitor 324 and one or more individual fixed-display results windows 326, implemented to provide information for the user. It is also noted that in the device 100 of FIG. 7, the RF transmitters 323 may include both transmission capabilities as well as receiving capabilities to thereby establish two-way communication between the device 100 and an external device, such as in the examples described herein.

Additional examples related to the device 100 of FIG. 7 can be found in the above-referenced U.S. Pat. No. 9,133,024.

In some embodiments, a wearable device having one or more features as described herein can be configured to sample blood of a user wearing the device. Based on such sampling of blood, one or more BAC-related measurements can be achieved as described herein.

FIG. 8 shows an example of a patch 100 configured to sample blood to allow BAC-related measurements to be performed by the patch 100. More particularly, the patch 100 of FIG. 8 can be configured to include a number of components implemented to allow, for example, one or more blood-related assays, measurements, etc., and notification of results of such assay(s)/measurement(s) to a monitoring device or system. Accordingly, the patch 100 can include a component configured to perform one or more blood-related assays/measurements, and communication components configured to, for example, receive control signals and/or transmit information resulting from the blood-related assay/measurement. In embodiments where such transmission of information is achieved in a wireless manner, the patch 100 can further include one or more antennas configured to support the receive and/or transmit operations. In some embodiments, some or all of the functionalities associated with the various components of the patch 100 can be controlled and/or supported by a processor.

In some embodiments, the patch 100 can include a support structure 330 configured to support various components, such as the example components described above. Such a support structure can include one or more layers, and can be implemented in a number of wearable forms. Examples of such a support structure and different wearable forms are provided in the above-referenced U.S. Pat. No. 9,133,024.

In some embodiments, biological fluid such as blood can be obtained by the patch 100, and the blood-related assay can be performed with such a biological fluid. In some embodiments, such as in the example of FIG. 8, the patch 100 can be configured to allow circulation of biological fluid therethrough, and sampling of the biological fluid for assay purpose can be obtained from such a circulating fluid. In the example of FIG. 8, such a circulating fluid is depicted as blood flowing in (331), and at least some of such blood flowing out (332) of the patch 100. In some embodiments, such in-flow and out-flow of blood into and out of the patch 100 can be supported by respective lancets or similar devices.

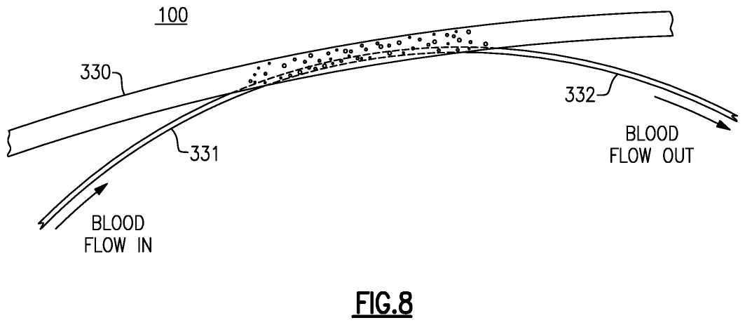

As described herein, a wearable device such as a patch having one or more features as described herein can include a communication component to facilitate transmission of information such as BAC-related assay/measurement data. FIG. 9 shows an example of a system that can be implemented to utilize such a communication functionality. For example, a patch 100 having one or more features as described herein is shown to be worn by a user 102. Information transmitted (e.g., in a wireless manner) is depicted as 180, and such information can be received by a monitor 106. Such a monitor can include a receiver circuit configured to process the received signal from the patch 100. The monitor 106 can further include a processor to facilitate, for example, notification of the assay/measurement data to the user and/or another person.

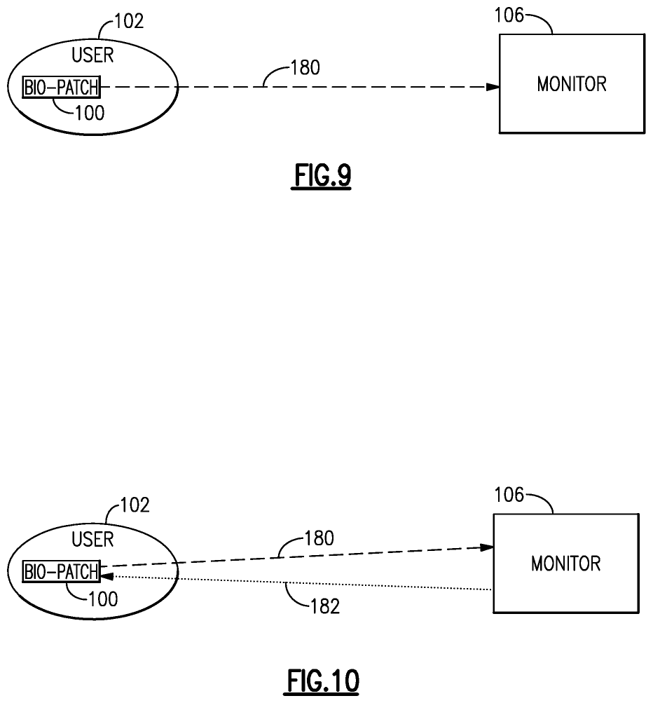

In some embodiments, a patch having one or more features as described herein can also include a receiver circuit to allow the patch to receive information such as instructions, diagnostics, etc. Accordingly, FIG. 10 shows an example of a system that can be implemented to utilize such transmit and receive functionalities. For example, a patch 100 having one or more features as described herein is shown to be worn by a user 102. Information transmitted (e.g., in a wireless manner) is depicted as 180, and such information can be received and processed by a monitor 106, similar to the example of FIG. 9.

In the example of FIG. 10, the patch 100 can also receive information (indicated as 182). Such received information can be achieved in a wireless mode, a wire mode, or any combination thereof. Although such information is depicted as being provided by the monitor 106, it will be understood that information provided to the patch 100 may or may not be from the same component (e.g., monitor 106 in FIG. 7).

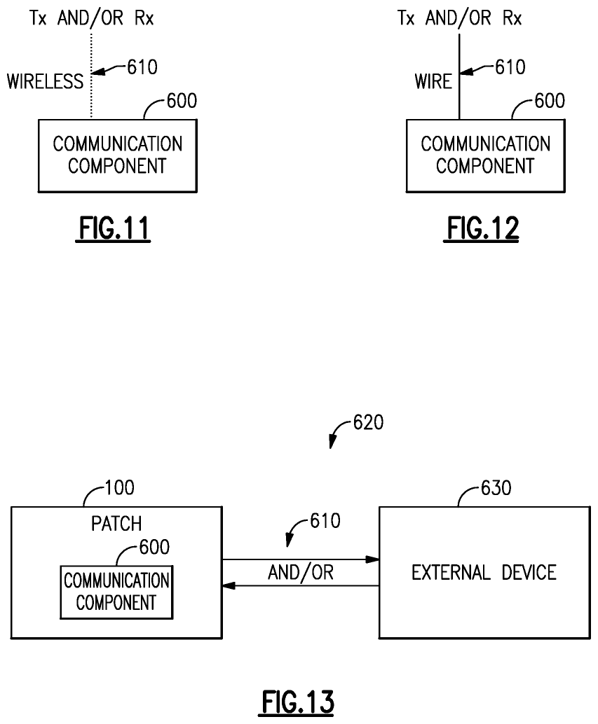

FIGS. 11 to 19 show examples of communications and/or system functionalities that can be implemented in a system having one or more wearable devices as described herein. For example, FIGS. 11 and 12 show that in some embodiments, a communication component 600 (e.g., 134 in FIGS. 5A and 5B) of a patch can be configured to provide a wireless communication (depicted as 610 in FIG. 11) with an external device, a wired communication (depicted as 610 in FIG. 12) with an external device, or some combination thereof. For the purpose of description of FIGS. 11 and 12, an external device can be another patch, a non-patch device, etc.

In some embodiments, in each of the examples of FIGS. 11 and 12, the wireless and/or wired communication link 610 can include a transmit (Tx) functionality (relative to the corresponding patch), a receive (Rx) functionality, or any combination thereof.

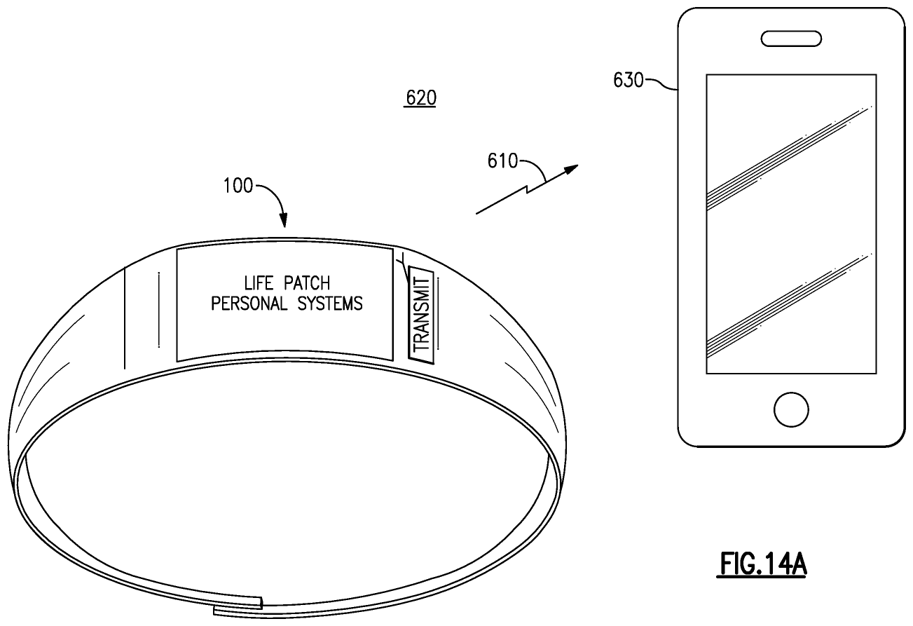

FIG. 13 shows a system 620 that can be formed with one or more wearable devices such as one or more patches 100 as described herein, and an external device 630. For the purpose of description of FIG. 13, it will be understood that the external device 630 is relative to the patch 100. Thus, if the external device 630 is another patch, then the patch 100 shown in FIG. 13 can be considered to be external to the other patch (630). As described in reference to FIGS. 11 and 12, it will be understood that the external device 630 can be a patch that may or may not be similar to the patch 100.

In the example of FIG. 13, the patch 100 is shown to include a communication component similar to the examples of FIGS. 11 and 12. Accordingly, the communication between the patch 100 and the external device 630 can include transmit and/or receive portions.

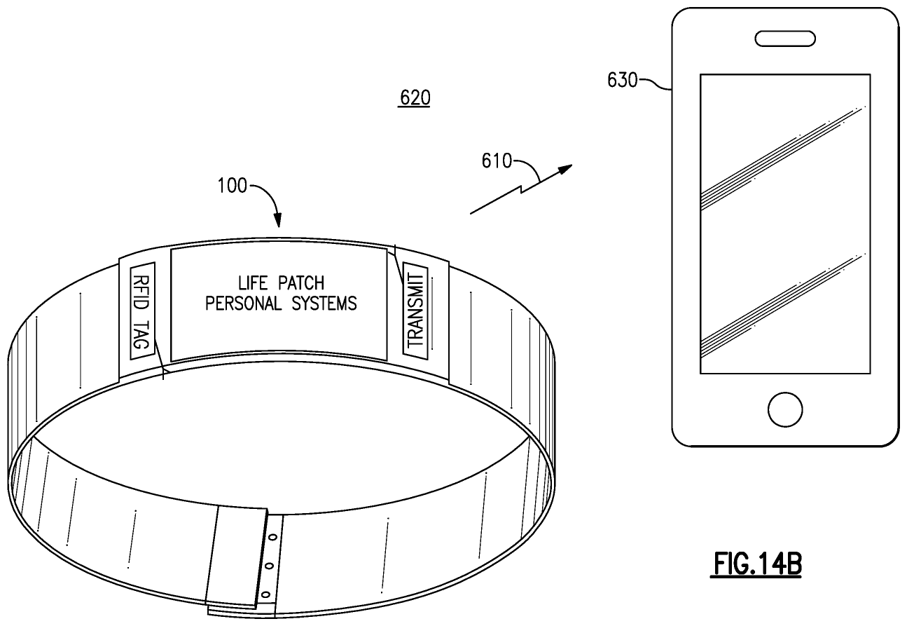

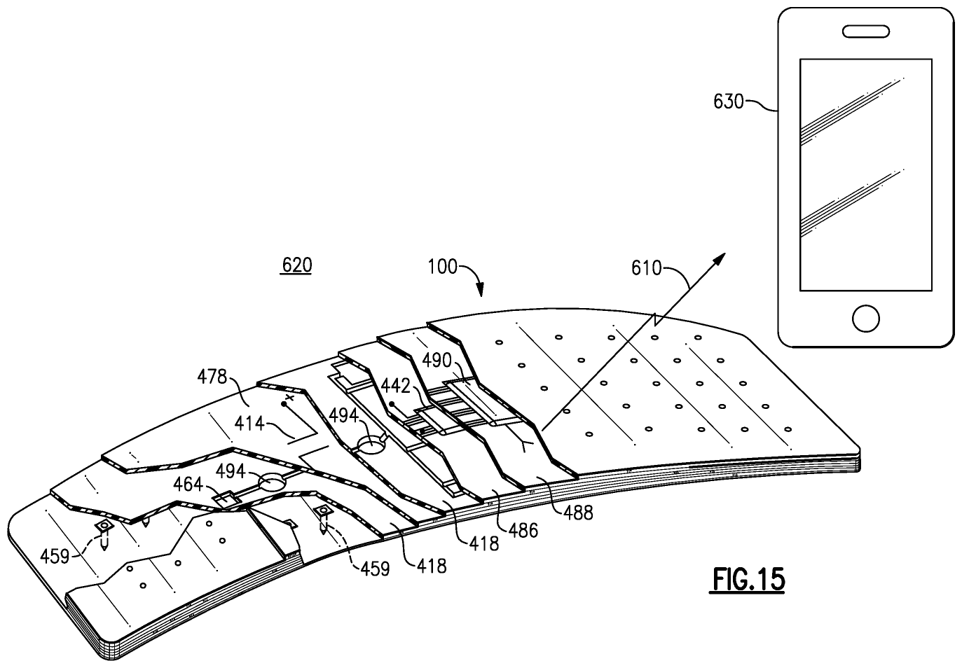

FIGS. 14 and 15 show examples of the system 620 of FIG. 13 implemented utilizing different types of wearable devices. For the purpose of such examples, the external device 630 of the system 620 is assumed to be a smartphone; however, it will be understood that similar system functionalities can also be achieved utilizing other types of external devices.

FIG. 14A shows that in some embodiments, a system 620 for monitoring alcohol content of a person can include a wearable device implemented as a ring or a ring-like device 100. Such a ring can be configured to provide various measurement and communication functionalities as described herein, so as to allow the ring 100 to send a wireless signal 610 to a smartphone 630. Such a signal can include information indicative of an alcohol content level of the person wearing the ring 100. The smartphone 630 can receive such a signal, and based on the received signal, perform one or more actions. Examples of such action(s) are described herein in greater detail.

FIG. 14B shows that in some embodiments, a system 620 for monitoring alcohol content of a person can include a wearable device implemented as a bracelet or a bracelet-like device 100, such as the example device described herein in reference to FIG. 6. Such a bracelet can be configured to provide various measurement and communication functionalities as described herein, so as to allow the bracelet 100 to send a wireless signal 610 to a smartphone 630. Such a signal can include information indicative of an alcohol content level of the person wearing the bracelet 100. The smartphone 630 can receive such a signal, and based on the received signal, perform one or more actions. Examples of such action(s) are described herein in greater detail.

FIG. 15 shows that in some embodiments, a system 620 for monitoring alcohol content of a person can include a wearable device implemented as a patch or a patch-like device 100. In the example of FIG. 15, the patch is shown to include a plurality of layers configured to perform analysis including blood content level analysis as described herein. More particularly, FIG. 15 shows minimally invasive tubules, lancets, or micro-probes 459 in a sample acquisition layer, reservoir openings 464 in layer 418, a draw-off reservoir or an initial sample collection chamber 494 also in layer 418, electrodes 414 in a results detection layer 478, a second fluid processing layer 418, a signal processor 442 in a signal processing layer 486, and a controller 490 in a logic and input/output controller layer 488. Additional details concerning the example patch 100 of FIG. 15 are provided in the above-referenced U.S. Pat. No. 9,133,024.

Referring to FIG. 15, the patch 100 can be configured to provide various measurement and communication functionalities as described herein, so as to allow the patch 100 to send a wireless signal 610 to a smartphone 630. Such a signal can include information indicative of an alcohol content level of the person wearing the patch 100. The smartphone 630 can receive such a signal, and based on the received signal, perform one or more actions. Examples of such action(s) are described herein in greater detail.

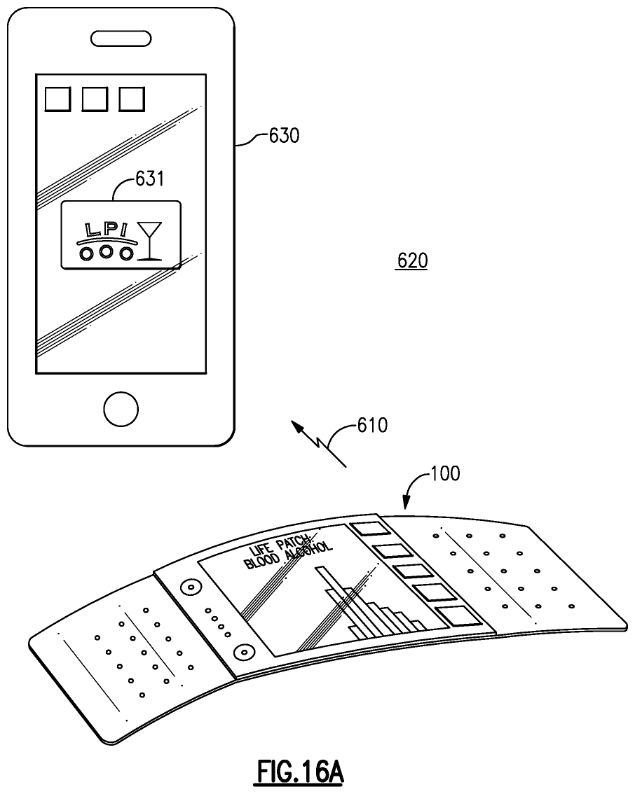

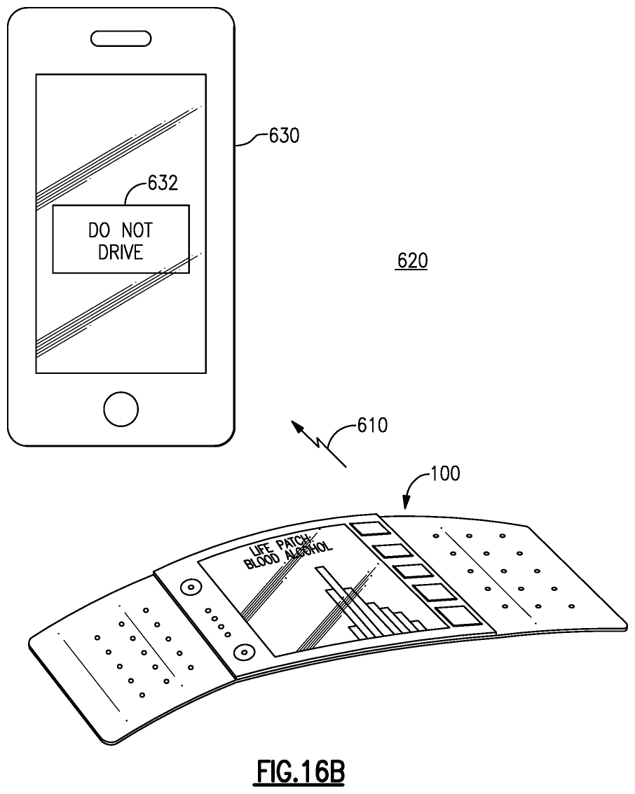

FIGS. 16A to 16C show examples of actions that can be performed by a smartphone 630 upon receipt of a signal 610 from a wearable device 100 in a system 620. In the examples of FIGS. 16A to 16C, the wearable device 100 is depicted as a patch; however, it will be understood that other wearable devices, including the examples of FIGS. 14A and 14B, can also be utilized.

Referring to FIG. 16A, the smartphone 630 is shown to include an application, a program, a software, and the like (referred to herein as an app) 631 operating therein. Such an app can be configured to process information received through the signal 610 and present different notices on the smartphone.

For example, FIG. 16B shows a situation where the patch 100 has detected sufficient level of alcohol content in the person wearing the patch 100, and thus, the signal 610 sent to the smartphone is indicative of such an impairment state. In response to receipt and processing of such a signal, the app 631 on the smartphone 630 can provide a notice 632 (e.g., “DO NOT DRIVE”) to prevent or discourage the impaired person from driving a vehicle.

In another example, FIG. 16C shows a situation where the level of alcohol content 633 in the person is determined (e.g., by the patch 100 in FIG. 16A) to be less than a threshold level, and thus, the signal 610 sent to the smartphone is indicative of such a state of the person. In response to receipt and processing of such a signal, the app 631 on the smartphone 630 can provide a notice 634 (e.g., “OK TO DRIVE”) to allow the person to operate a vehicle.

In the examples of FIGS. 16B and 16C, the notices provided on the smartphone 630 include informational notices presented to a person in close proximity to the smartphone 630. FIG. 17 shows that in some embodiments, the system 620 of FIG. 16A can further include a second external device 623 such as a second smartphone to provide an extended configuration 621. In the example of FIG. 17, the first smartphone 630 is capable of communicating with the second smartphone 623 (which may be close by or far away). Thus, a second signal 622 is shown to be sent from the first smartphone 630 to the second smartphone 623; and information contained in the second signal 622 can be based on the action of the first smartphone 630 which in turn is based on the signal (e.g., 610 in FIG. 16B) provided by the patch 100.

For example, suppose that the app in the first smartphone 630 generates a notice 632 (e.g., “DO NOT DRIVE”) to prevent or discourage the impaired person from driving a vehicle, as described in reference to FIG. 16B. The smartphone 630, through the app or another app, can induce sending of the signal 622 to the second smartphone 623 to allow another person associated with the second smartphone 623 to assist the impaired person associated with the first smartphone 630.

For example, suppose that a first member of a household is associated with the first smartphone 630, and he/she consumes alcohol while out of the house and while wearing a patch as described herein. If the first person becomes impaired, such a state can be detected by the patch, and the first smartphone 630 can provide a notice 632 (e.g., “DO NOT DRIVE”) to prevent or discourage the impaired first person from driving a vehicle. A second member of the household at the house or elsewhere, and not with the first person, can be informed of the situation on his/her smartphone 623 based on the information 622 received from the first smartphone 630. Based on the information provided on the second smartphone 623, the second person can provide assistance to the first person to safely transport the first person. For example, a ride request can be included in the information provided on the second smartphone 623, and based on such a request, the second person can drive to the location of the first smartphone 630 (e.g., based on location information provided by the first smartphone), or arrange for a ride through ride-sharing, taxi, etc.

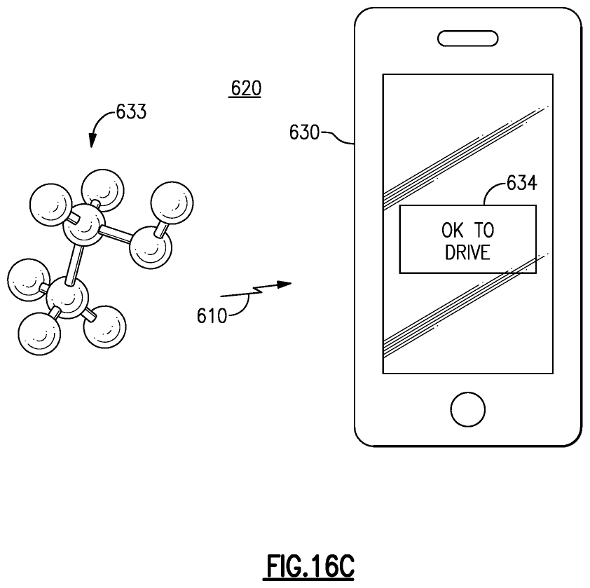

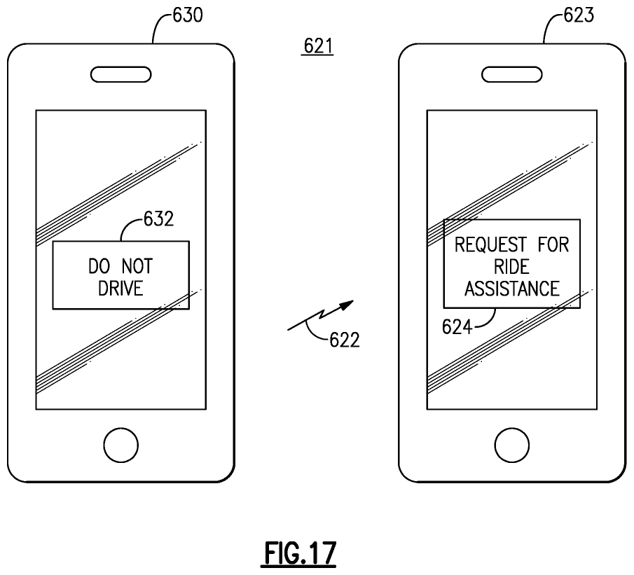

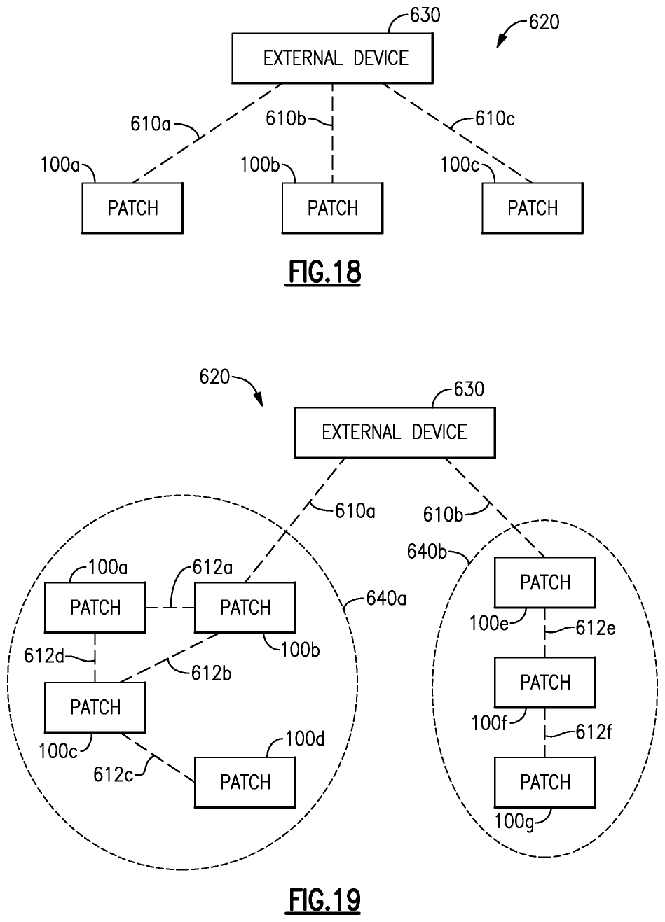

FIG. 18 shows that in some embodiments, the system 620 of FIG. 13 can include a plurality of patches 100 that communicate with a common external device. For example, a system 620 of FIG. 18 is shown to include a plurality of patches 100a, 100b, 100c and an external device 630. More particularly, the first patch 100a can be in communication (610a) with the external device 630, the second patch 100b can be in communication (610b) with the external device 630, and the third patch 100c can be in communication (610c) with the external device 630. In some embodiments, such an external device can be configured to, for example, coordinate operations of the patches (100a, 100b, 100c), collect data from the patches, etc. In some embodiments, the external device 630 can be configured to communicate with another device at a similar level, with another device at a higher level, or any combination thereof.

FIG. 19 shows that in some embodiments, the system 620 of FIG. 13 can include a plurality of patches 100 that can communicate with each other, and/or with an external device. For example, a first group (640a) of patches and a second group (640b) are shown to be included in a system 620, and generally in communication with an external device 630. More particularly, the first group 640a is shown to include four example patches 100a, 100b, 100c, 100d, and the second group 640b is shown to include three example patches 100e, 100f, 100g. Such first and second groups 640a, 640b of patches can be grouped based on, for example, physical proximity/separation, different functionalities, etc.

In some embodiments, within a given group, each of the plurality of patches can communicate directly with the external device 630, through a representative patch, or some combination thereon. For example, for the first group 640a, the patches 100a and 100b are shown to have a communication link 612a; the patches 100a and 100c are shown to have a communication link 612d; the patches 100c and 100d are shown to have a communication link 612c; and the patches 100c and 100b are shown to have a communication link 612b. Further, the patch 100b is shown to be a representative communication member and be in communication (610a) with the external device 630.

In another example, for the second group 640b, the patches 100e and 100f are shown to have a communication link 612e; and the patches 100f and 100g are shown to have a communication link 612f. Further, the patch 100e is shown to be a representative communication member and be in communication (610b) with the external device 630.

In some embodiments, the communication links between the patches within a given group can be based on, for example, relative proximity/distance among the users wearing the respective patches, some hierarchy of the users and/or patches, or some combination thereof. In some embodiments, the communication links between the patches can be configured as a mesh network, or be based on such a network.

In some embodiments, a system of patches as described herein (e.g., in reference to FIGS. 11-13, 18 and 19) can provide a system level information that may not be available from an individual patch.

In some implementations, a patch having one or more features as described herein can be utilized in a system for preventing or reducing the likelihood of a person from driving a vehicle while in an impaired state. Such a person in the impaired state is sometimes referred to as a drunk person or a drunk driver. For the purpose of description, a driver can include a person who is actually driving a vehicle, or a person who is attempting to drive a vehicle.

Although various examples are described in the context of alcohol based impairment, it will be understood that one or more features of the present disclosure can also be implemented in systems dealing with other forms of impairments, including, for example, narcotics.

FIG. 20 shows an example system 700 where a driver 102 is wearing a patch 100 having one or more features as described herein. Such a patch can communicate with a vehicle controller such as a vehicle electronic control unit (ECU) 710. Such an ECU can be configured to control a number of functionalities of a vehicle, including, for example, engine start (or motor enable in an electric vehicle), vehicle entry, etc.

In some embodiments, the communication between the patch 100 and the vehicle ECU 710 can be achieved by a substantially direct communication link (depicted as 712), or through one or more compliance devices 702 (depicted as communication links 704a and 704b). For the former example where a direct communication link 712 is utilized, the vehicle ECU 710 can be configured to provide one or more compliance functionalities as described herein to inhibit or reduce the likelihood of a driver operating the vehicle. For the latter example where intermediate compliance device(s) is/are utilized, one or more compliance functionalities can be implemented in the intermediate compliance device(s) 702, in the vehicle ECU 710, or some combination thereof.

For example, FIG. 21 shows a compliance logic circuit 720 that can be implemented in the vehicle ECU 710 of FIG. 20. In the example, an engine start logic can be implemented where one or more vehicle conditions need to be satisfied, and one or more driver related conditions also need to be satisfied. For the purpose of description, it will be assumed that a state of “1” (or high) is a good condition, and a state of “0” is a bad condition (or low).

For example, suppose that “Vehicle condition 1” depicted in FIG. 14 is the presence or absence of a key fob, with “1” being “Yes” and “0” being “No”; and “Vehicle condition 2” is the state of the brake pedal, with “1” being “Applied” and “0” being “Not applied.” If any of such vehicle conditions is “0,” the “Enable start” output will be a “0” and the vehicle will not start.

In some embodiments, either or both of “Driver condition” state and “Driver authentication” state can be provided to affect the “Enable start” output state. For example, if a driver is determined to be impaired by a patch (worn by the driver), “Driver condition” can be “0”; and if the driver is determined to be not impaired by the patch, “Driver condition” can be “1.” In another example, if a driver is determined to be wearing the foregoing patch, “Driver authentication” can be “1”; and if the driver is determined to be not wearing the patch, “Driver condition” can be “0.”

It is noted that in some embodiments, the foregoing functionalities related to “Driver condition” state and “Driver authentication” state can be implemented outside of the vehicle ECU 710. For example, either or both of such functionalities related to “Driver condition” state and “Driver authentication” state can be implemented in the one or more intermediate compliance devices 702. In such a configuration, information sent to the vehicle ECU 710 can be driver-specific information such as in the example of FIG. 21, a simple Go/No-go status signal without any driver information (e.g., 1=allow engine start, or 0=do not allow engine start), or some combination thereof.

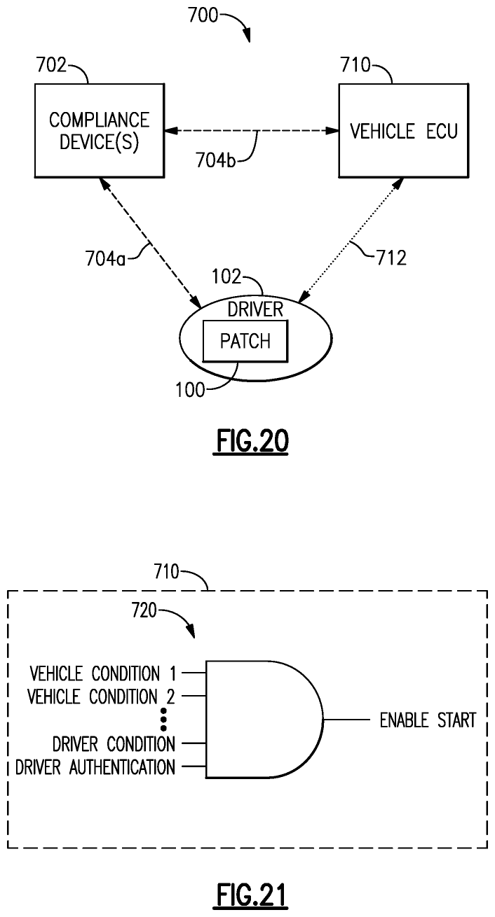

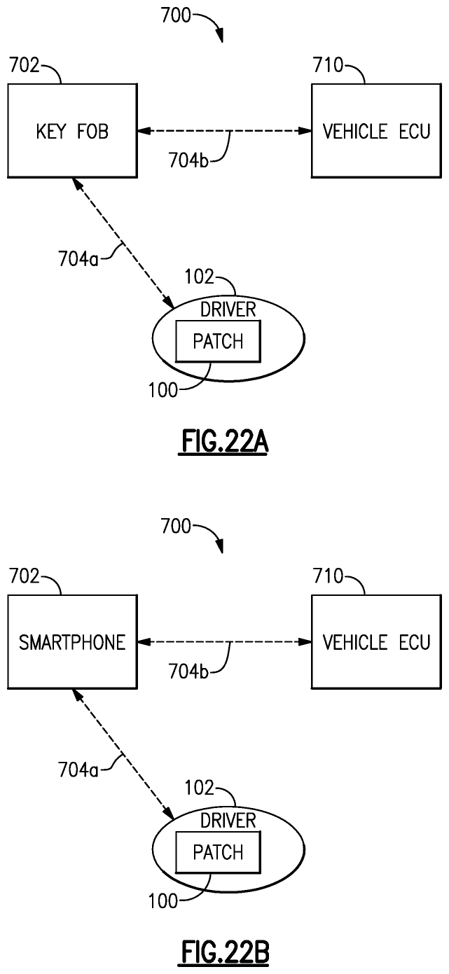

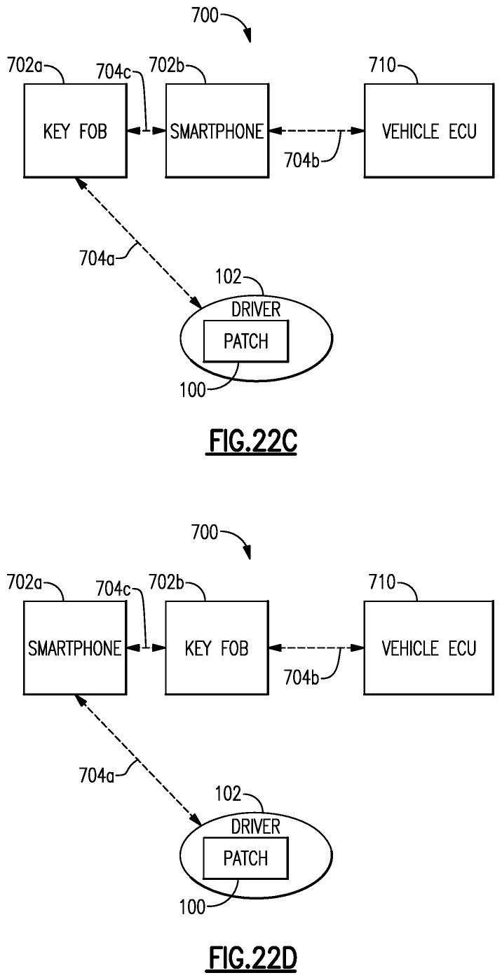

FIGS. 22A to 22D show non-limiting examples of one or more intermediate compliance devices that can be implemented in the system of FIG. 20. For example, FIG. 22A shows that in some embodiments, a key fob 702 can be configured as an intermediate compliance device. Such a key fob can communicate with a vehicle ECU 710 (through a communication link 704b) to provide typical key fob functionalities (e.g., door lock/unlock, remote engine start, keyless engine start, etc.), as well as some or all of one or more compliance functionalities as described herein.

In the example of FIG. 22A, the key fob 702 is shown to communicate with a patch 100 worn by a driver 102, through a communication link 704a. Such a communication link can be a passive communication whenever the patch 100 is within some range of the key fob 702, be an on-demand communication (e.g., when a button is pressed on the key fob 702), be initiated by the patch 100, be initiated by the key fob 702, or some combination thereof. In some embodiments, the communication link can be achieved through an RFID (radio-frequency identification) circuit implemented in the patch 100 and supported by the key fob 702.

In another example, FIG. 22B shows that in some embodiments, a portable wireless device such as a smartphone 702 can be configured as an intermediate compliance device. Such a smartphone can communicate with a vehicle ECU 710 (through a communication link 704b) to provide typical smartphone functionalities (e.g., music, handsfree phone, etc.), as well as some or all of one or more compliance functionalities as described herein.

In the example of FIG. 22B, the smartphone 702 is shown to communicate with a patch 100 worn by a driver 102, through a communication link 704a. Such a communication link can be a passive communication whenever the patch 100 is within some range of the smartphone 702, be an on-demand communication (e.g., when an app is activated on the smartphone 702), be initiated by the patch 100, be initiated by the smartphone 702, or some combination thereof. In some embodiments, the communication link can be achieved through an RFID (radio-frequency identification) circuit implemented in the patch 100 and supported by the smartphone 702.

In yet another example, FIG. 22C shows that in some embodiments, more than one intermediate compliance devices can be utilized. For example, a patch 100 worn by a driver 102 can communicate with a key fob 702a through a communication link 704a, and the key fob 702a can communicate, at least for some or all of driver's compliance related information, to a vehicle ECU 710 through a portable wireless device such as a smartphone 702b. More particularly, the key fob 702a can communicate with the smartphone 702b through a communication link 704c, and the smartphone 702b can communicate with the vehicle ECU 710 through a communication link 704b. In the example of FIG. 22C, one or more compliance functionalities as described herein can be provided by the key fob 702a, by the smartphone 702b, or some combination thereof.

In yet another example, FIG. 22D shows that in some embodiments, more than one intermediate compliance devices can be utilized. For example, a patch 100 worn by a driver 102 can communicate with a portable wireless device such as a smartphone 702a through a communication link 704a, and the smartphone 702a can communicate, at least for some or all of driver's compliance related information, to a vehicle ECU 710 through a key fob 702b. More particularly, the smartphone 702a can communicate with the key fob 702b through a communication link 704c, and the key fob 702b can communicate with the vehicle ECU 710 through a communication link 704b. In the example of FIG. 15D, one or more compliance functionalities as described herein can be provided by the smartphone 702a, by the key fob 702b, or some combination thereof.

It is noted that some vehicles may be equipped with compliance devices such as breathalyzer implemented to have a driver pass a breathalyzer test prior to operation of a corresponding vehicle. In some embodiments, a patch having one or more features as described herein can be implemented to replace such a compliance device (e.g., remove the need for a breathalyzer and utilize the patch), to be used as an alternate option of demonstrating compliance (e.g., use either the breathalyzer or the patch), or to be used with such a compliance device. For example, in a setting where both of the compliance device (e.g., breathalyzer) and the patch are utilized, compliance can be demonstrated by passing both of the breathalyzer and the patch.

For the purpose of description, it will be understood that a vehicle ECU can be implemented as a single device (e.g., a single module), as an assembly of a plurality of devices to provide one or more control functionalities for the vehicle, or some combination thereof.

FIG. 23 shows that in some embodiments, one or more patches having one or more features as described herein can be provided in a packaged format 882 for easier use. Such a packaged format of patch(es) can be included in, for example, a packaged product 880. In some embodiments, the packaged product 880 can also include an instruction 884 such as a printed instruction. Such an instruction can provide information on, for example, proper and/or recommended application and use of the included patch(es).

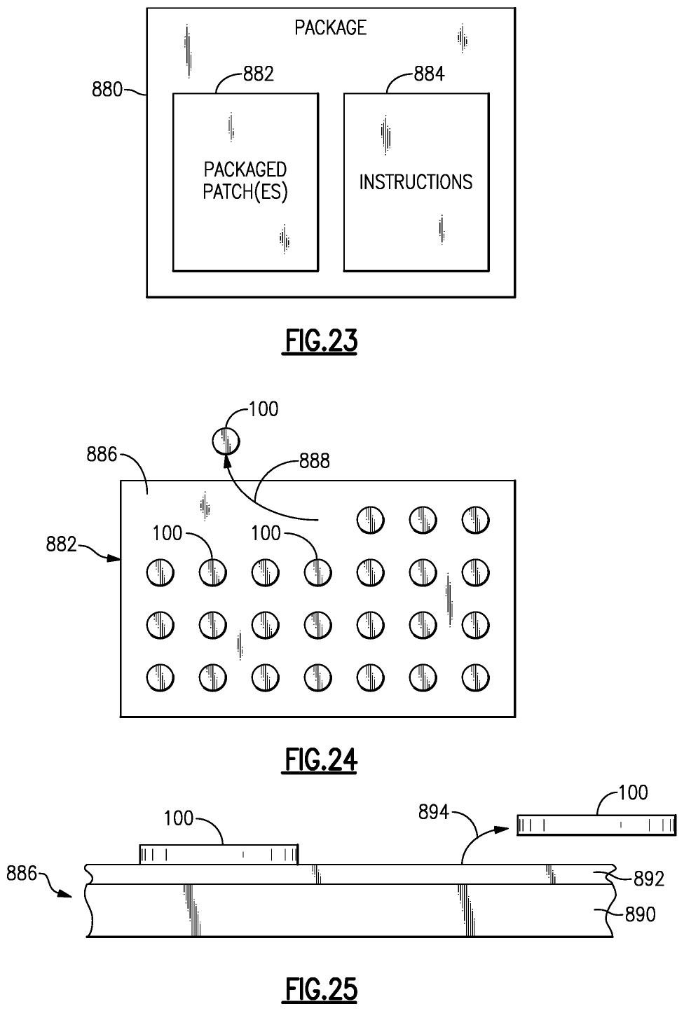

FIG. 24 shows an example of a packaged format 882 having a support sheet 886 with a plurality of patches 100 secured thereto. Such number of patches can allow one to remove (arrow 888) a patch 100 from the support sheet 886 for use. For example, one patch can be utilized during an event where alcohol is, or likely will be, consumed.

FIG. 25 shows an enlarged side sectional view of an example support sheet 886 that can be utilized to hold (until removal) a plurality of patches, similar to the example of FIG. 24. In some embodiments, the support sheet 886 can include a base layer 890 (e.g., paper, plastic, etc.) and a release layer 892. The release layer 892 can be secured to the base layer 890, and be configured to securely hold the patches 100 thereon during transport and storage phases. Assuming that a patch includes an adhesive layer for application onto the skin of a user, the release layer can further be configured to allow the patch to be removed (e.g., peeled off) cleanly for application onto the user. In the example of FIG. 25, such removal of the patch 100 from the release layer 892 is depicted as an arrow 894.

In many applications, it is highly desirable to be able to determine that a compliance process involving a patch indeed corresponds to a proper person (e.g., a person wearing the patch) attempting to operate a vehicle. For the purpose of description, it will be understood that an authentic driver is a person wearing a patch that is providing information about the person (e.g., BAC level of the person) and attempting to operate a vehicle. Accordingly, driver authentication can refer to whether a person is an authentic driver or not.

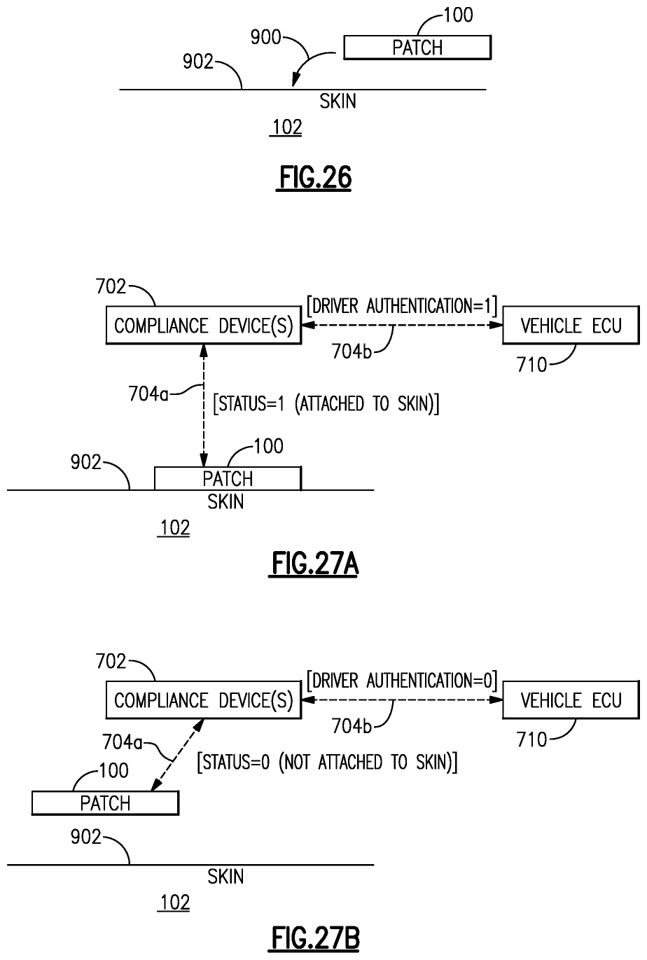

Described herein are examples of how driver authentication can be implemented. For example, suppose that a person attaches a patch to his/her skin according to the example of FIG. 25. Such a patch application step is depicted in FIG. 26, where a patch 100 having one or more features as described herein is placed (indicated as 900) on a skin 902 of a person 102. Such a patch application step can occur before or during alcohol consumption, and preferably prior to an attempt at operation of a vehicle. For the purpose of description, such a person wearing the patch is an authentic driver and has a driver authentication status of “1.”

There may be a situation where a person may attempt to circumvent a compliance setting as described herein. One example is where a patch worn by the original person is transferred to another person in an attempt to have the patch measure lower BAC level below some threshold value. Such another person is likely a person that has consumed little or no alcohol. For the purpose of description, such another person now wearing the transferred patch is not an authentic driver and has a driver authentication status of “0.”

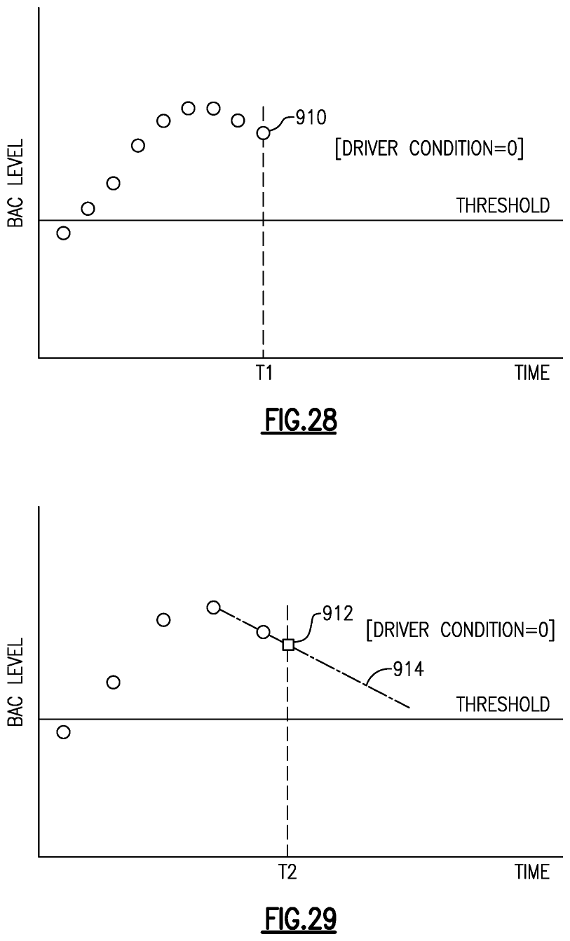

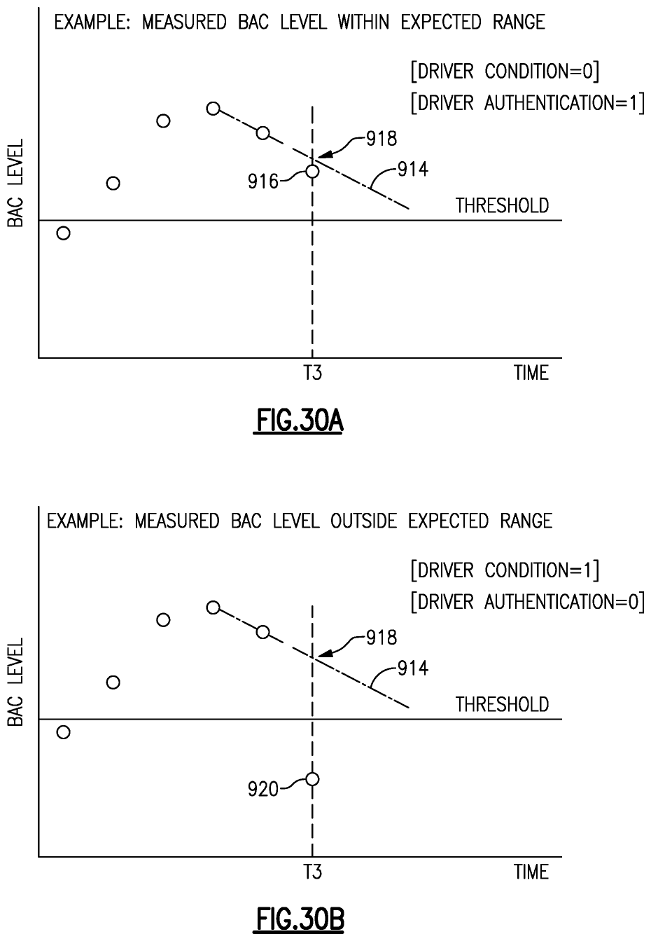

FIGS. 27A and 27B show an example where authentication of a patch user, or driver authentication, can be determined. In some embodiments, a patch 100 having one or more features as described herein can be configured to determine whether the patch is attached to a skin 902 or detached from the skin 902 of a user. For example, a status bit can be set to “1” if the patch 100 detects that it is engaged to the skin 902, and be set to “0” if the patch 100 is not engaged to the skin 102. In some embodiments, the patch 100 can be configured such that once the status bit changes from “1” to “0,” the bit cannot be changed back to “1,” even if re-attached to a skin (e.g., skin of the original person or someone else).

Referring to FIG. 27A, when the patch 100 remains attached to the skin 902 of the user 102, the attached status is “1.” Such a status can be provided to an intermediate compliance device 702 through a communication link 704a, and accordingly, a driver authentication status of “1” can be provided to a vehicle ECU 710 through a communication link 704b.

Referring to FIG. 27B, when the patch 100 is removed from the skin 902 of the user 102, the attached status is “0.” Such a status can be provided to the intermediate compliance device 702 through the communication link 704a, and accordingly, a driver authentication status of “0” can be provided to the vehicle ECU 710 through the communication link 704b.

In the example of FIGS. 27A and 27B, the communication link between the patch 100 and the vehicle ECU 710 is assumed to include an intermediate device 702. It will be understood that a driver authentication status can be provided from the patch 100 to the vehicle ECU 710 in a direct manner, similar to the examples of FIG. 20.

One can see that in the example of FIGS. 27A and 27B, authentication of a driver can be implemented by determining whether a patch remains attached or not. In some embodiments, such an authentication method can depend on how a patch itself is configured.

FIGS. 28 to 30 show that in some embodiments, driver authentication can be achieved even if a patch cannot detect the attached/detached status. For example, FIG. 28 depicts an operation of a system that utilizes a patch having one or more features as described herein. More particularly, suppose that such a system samples blood alcohol content (BAC) levels over time. In FIG. 28, such BAC levels are indicated as circle datapoints, and at time T1, the BAC level 910 is shown to be above a threshold value. Accordingly, condition of a person wearing the patch has a status of “0” (Driver condition=“0”).

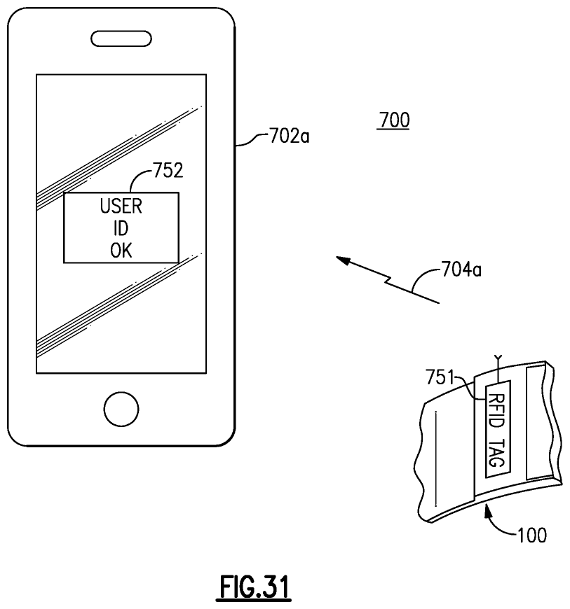

In some embodiments, a patch and/or a related system can be configured to predict or estimate (e.g., by interpolation or extrapolation) a BAC level at a time away from a measurement time (e.g., T1 in FIG. 28). For example, such prediction/estimation can be based on a trend obtained from measurements. More particularly, the measurement trend in FIG. 29 can (e.g., based on decline in BAC levels combined with time information) can indicate that the person wearing the patch likely stopped consuming alcohol, and the BAC level is declining at a predictable or estimated rate (indicated as 914). Accordingly, if an estimate of BAC level is desired at time T2, and there is no measurement at such a time, BAC level 912 can be estimated.

In the examples of FIGS. 28 and 29, it is noted that the Driver condition status is “0” (i.e., BAC level above the threshold level) in each case, based on measurement or estimation/prediction. In some embodiments, such estimation/prediction functionality can be utilized to identify a person as an authentic driver or a non-authentic driver.

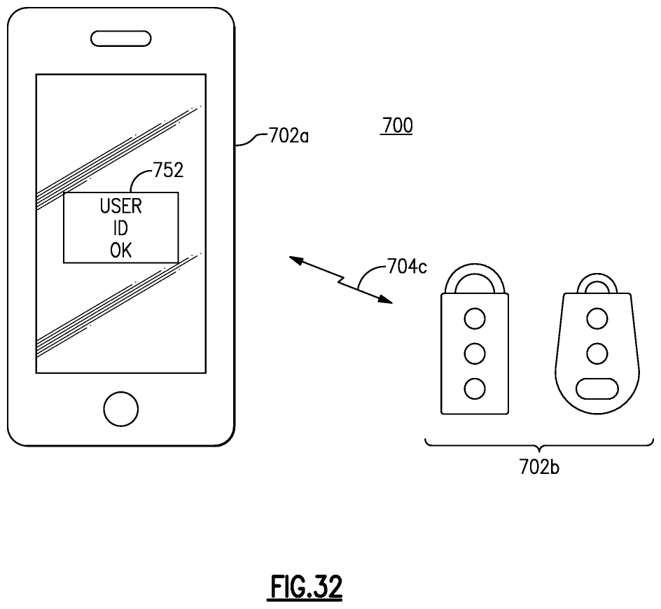

For example, and referring to FIGS. 30A and 30B, suppose that a patch is being utilized by a person as described in reference to FIG. 29. At time T3, if that person attempts to operate a vehicle, a measured BAC value 916 can be obtained and compared to an expected value 918. As described herein, such an expected value can be obtained by, for example, extrapolation or interpolation (914). If the difference between the measured value 916 and the expected value 918 is within a selected range, one can be fairly certain that the patch is indeed being used by the original person. Accordingly, Driver authentication status can be “1.” In the example of FIG. 30A, however, the measured BAC value 916 is above the threshold level; thus, Driver condition status is determined to be “0”; and the original person will not be able to operate the corresponding vehicle.

Referring to FIG. 30B, suppose that the patch is removed from the original person and attached to another person who has consumed little or no alcohol. Accordingly, at the example time T3, a measured BAC value 920 will be below the threshold value, and Driver condition status can be “1.” However, since the difference between the measured value 920 in FIG. 30B and the expected value 918 is outside the selected range, one can be fairly certain that the patch is not being used by the original person. Accordingly, Driver authentication status can be “0,” and the original person will not be able to operate the corresponding vehicle.

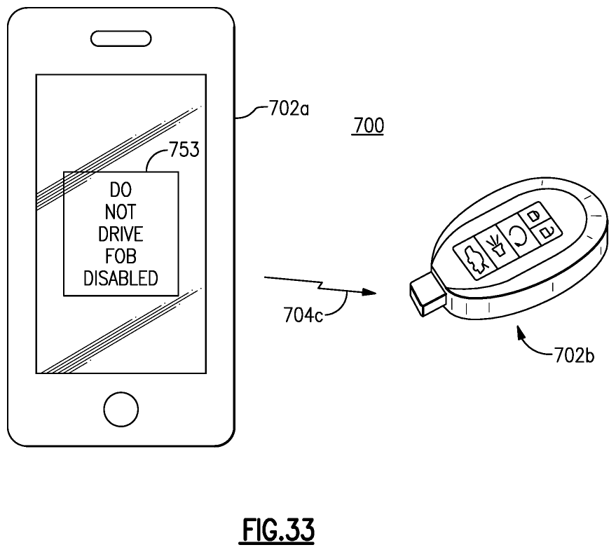

FIGS. 31 to 36 show various examples of a system 700, such as the system 700 of FIG. 22D, that utilizes an external device such as a smartphone 702a and one or more key fobs 702b associated with respective one or more vehicles. As described herein such a system can prevent an impaired person (wearing a wearable device as described herein) from operating a vehicle associated with a key fob linked to the smartphone 702b.

FIG. 31 shows that a wearable device (such as a patch) 100 as described herein can include an RFID tag 751, and such a circuit can be utilized to provide a signal 704a to the smartphone 702a to provide, for example, an ID verification 752 between the smartphone 702a and the patch 100 (and thus the person wearing the patch 100).

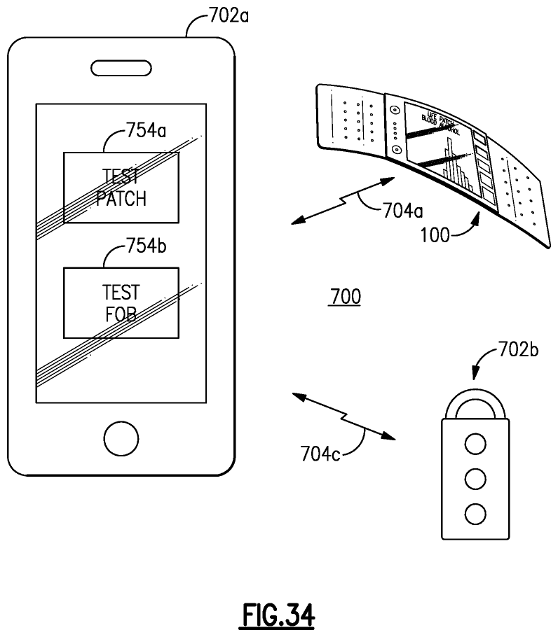

FIG. 32 shows that the smartphone 702a can further communicate with one or more key fobs 702b associated with the person identified in the example of FIG. 31. For example, if the person is associated with only one vehicle, the smartphone 702a can form a link 704c with each key fob associated with that single vehicle. In another example, if the person has access to a plurality of vehicles, then the smartphone 702a can form a link 704c with each key fob associated with each of the plurality of vehicles.

FIG. 33 shows a situation where the system 700 of FIG. 32 has already established a link between a smartphone 702a and a key fob 702b. Based on a detection functionality of a patch worn by a corresponding person, an app in the smartphone 702a has determined that the person is impaired. Accordingly, the app is shown to display a warning 753 to the impaired person (e.g., “DO NOT DRIVE FOB DISABLED”). In addition to such a warning, the app can cause a signal 704c to be sent from the smartphone 702a to the key fob 702b, and the key fob 702b can be disabled based on the signal 704c. Accordingly, the key fob 702b will not allow, for example, starting of the corresponding vehicle.

It is noted that in the example of FIG. 33, the smartphone 702a can send an enable signal to the key fob 702b upon determination that the previously impaired person is no longer impaired. Based on such an enable signal, the key fob 702b can be utilized to operate the vehicle.

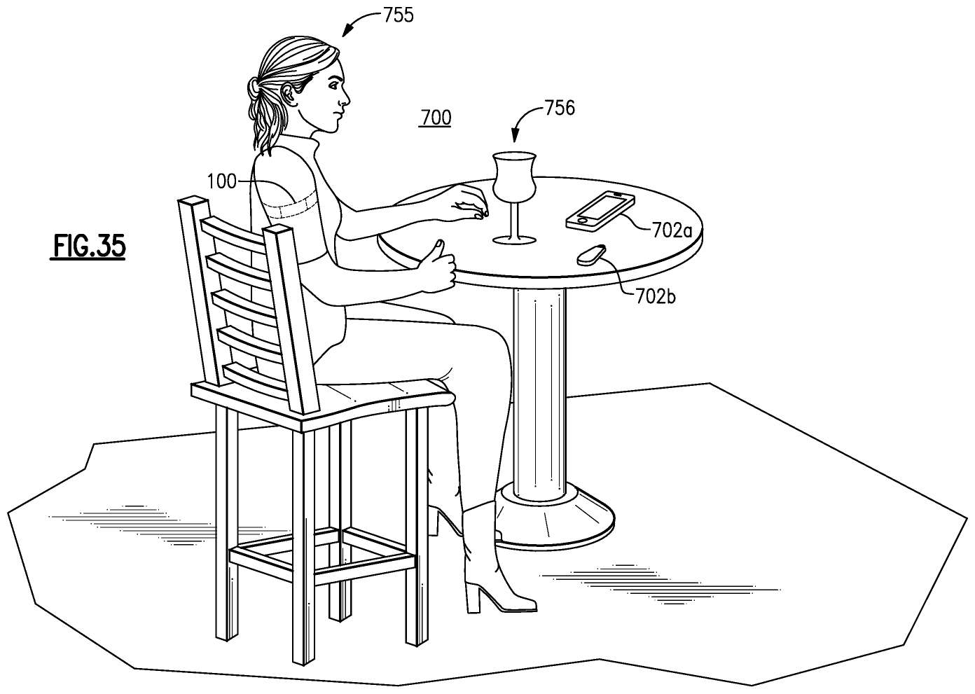

FIG. 34 shows that in some embodiments, a system 700 (such as the system of FIGS. 31 to 33) can include a diagnostic or test functionality. For example, a smartphone 702a can be configured to provide a test functionality with a patch 100. Such a test can be initiated by a user interface that provides a user input 754a, and can include, for example, communication link test, power level test, and/or other operating parameter or condition test with respect to the patch 100. In another example, the smartphone 702a can be configured to provide a test functionality with a key fob 702b. Such a test can be initiated by a user interface that provides a user input 754b, and can include, for example, communication link test, power level test, and/or other operating parameter or condition test with respect to the key fob 702b.

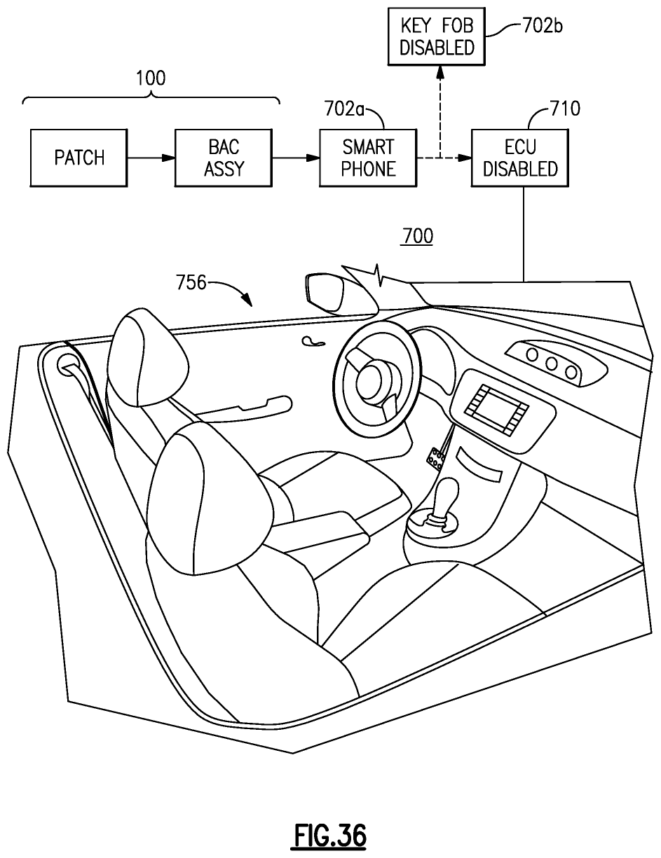

FIGS. 35 and 36 show an example of how a system 700 (such as the system of FIGS. 31 to 34) can be beneficial useful when a person 755 enjoys one or more alcoholic beverages 756. The person may or may not be aware of the impairment state resulting from consumption of such beverages. However, and as depicted in FIG. 35, a patch 100 worn by the person 755 can monitor the alcohol content as described herein, and such monitoring information can be transmitted to a smartphone 702a which is typically in possession of the person 755 (or nearby). Assuming that the person 755 drove to the drinking location in his/her own vehicle, a key fob 702b is also in possession of the person 755 (or nearby). Accordingly, the system 700 can allow the impaired state of the person 755 to be detected as described herein.

As also described herein, the detected impairment state can allow the system 700 to disable the operation of the vehicle, and such a disabling process is depicted in FIG. 36. As described herein, a patch 100 can be configured to detect a blood alcohol content (BAC) level, and information indicative of such a level can be provided to a monitoring device such as a smartphone 702a. In response to the impaired state information, the smartphone 702a can provide a signal that disables, for example, the ECU 710 of the vehicle 756. Such disabling signal can be provided directly to the vehicle 756, to the key fob 702b, or some combination thereof.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. A wearable patch comprising:
a patch structure having one or more layers and configured to allow the patch to be worn by a user;
an analyzer component implemented on or at least partially within the patch structure, and configured to measure alcohol content level in the user; and
an interface component implemented on or at least partially within the patch structure, and in communication with the analyzer component, the interface component configured to provide a notification based on the measured alcohol content.

2. The wearable patch of claim 1 wherein the patch structure is configured to allow the patch to be worn on or near a skin of the user.

3. The wearable patch of claim 3 wherein the patch structure is configured to allow the patch to be worn directly on the skin of the user.

4. The wearable patch of claim 1 wherein the interface component is configured to alert either or both of the user and another person when the measured alcohol content exceeds a selected level.

5. The wearable patch of claim 1 wherein the interface component includes a communication circuit configured to send information about the measured alcohol content.

6. The wearable patch of claim 5 wherein the communication circuit is configured to receive information.

7. (canceled)

8. (canceled)

9. (canceled)

10. A kit for monitoring alcohol content of a user, the kit comprising:
a wearable patch that includes a patch structure having one or more layers and configured to allow the patch to be worn by the user, the wearable patch further including an analyzer component implemented on or at least partially within the patch structure, and configured to measure alcohol content level in the user, the wearable patch further including an interface component implemented on or at least partially within the patch structure, and in communication with the analyzer component, the interface component configured to provide a notification based on the measured alcohol content; and
a printed instruction configured to facilitate use of the wearable patch.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. A system for monitoring impairment state of a person, the system comprising:
a patch configured to be attached to a skin of the person and obtain a measurement indicative of an impairment state of the person, the patch further configured to transmit information representative of the measurement; and
a monitor external to the patch, the monitor configured to receive the information from the patch through a communication link and generate an output based on the information.

24. The system of claim 23 wherein the communication link between the patch and the monitor is a direct link implemented with a wireless signal.

25. The system of claim 23 wherein the communication link between the patch and the monitor includes an intermediate component such that a first link provides communication between the patch and the intermediate component and a second link provides communication between the intermediate component and the monitor.

26. The system of claim 25 wherein the first link between the patch and the intermediate component is implemented with a wireless signal, and the second link between the intermediate component and the monitor is implemented with a wireless signal.

27. The system of claim 23 wherein the patch includes a radio-frequency identification (RFID) circuit configured to receive an interrogation signal and transmit the information in response to receipt of the interrogation signal.

28. The system of claim 23 further comprising a vehicle electronic control unit configured to receive the output of the monitor and either enable or disable an operation of a corresponding vehicle.

29. The system of claim 28 wherein the operation of the vehicle includes starting of an internal combustion engine or energizing of an electric drive motor.

30. The system of claim 28 wherein the output of the monitor is based on the measurement.

31. The system of claim 28 wherein the output of the monitor is based on an estimation based on a plurality of measured values related to the impairment state of the person.

32. The system of claim 28 wherein the output of the monitor is based on determination of whether or not the patch remains attached to the skin of the person.

33. The system of claim 32 wherein the patch is configured to sense detachment of the patch from the skin of the person.

34. The system of claim 32 wherein the determination of whether or not the patch remains attached to the skin of the person is based on a comparison of a measured value related to the impairment state of the person with an expected value.

35. The system of claim 23 wherein the measurement includes a blood alcohol content (BAC) level.
